(19) United States
(12) Patent Application Publication
(10) Pub. No.: US 2010/0028439 A1
(43) Pub. Date: Feb. 4, 2010

(54) Nanoparticulate stabilized anti-hypertensive compositions

(75) Inventors: Scott Jenkins, Downingtown, PA (US); Gary G. Liversidge, West Chester, PA (US)

Correspondence Address: Elan Drug Delivery, Inc. c/o Foley & Lardner, 3000 K Street, N.W., Suite 500, Washington, DC, 20007-5109, US

(73) Assignee: Elan Pharma International Limited

(21) Appl. No.: 11/437,833

(22) Filed: May 22, 2006

Related U.S. Application Data
(60) Provisional application No. 60/683,761, filed on May 23, 2005.

(51) Int. Cl. A61K 31/554; A61K 9/14; A61P 9/00

(52) U.S. Cl. 424/489; 514/211.03

(57) ABSTRACT

The present invention is directed to anti-hypertensive compositions comprising a nanoparticulate temocapril, or a salt or derivative thereof, having improved bioavailability. The nanoparticulate temocapril particles of the composition have an effective average particle size of less than about 2000 nm and are useful in the treatment of hypertension and related diseases.

### CROSS-REFERENCE TO RELATED APPLICATIONS

This application claims the benefit under 35 U.S.C. §119(e) to U.S. provisional application No. 60/683,761, filed on May 23, 2005, the entire contents of which are incorporated herein by reference.

### FIELD OF INVENTION

The present invention relates generally to compounds and compositions useful in the treatment and prevention of diseases and disorders that may include hypertension and other blood pressure-related diseases. More specifically, the invention relates to nanoparticulate compositions that include a nanoparticulate thiazepine compound, such as temocapril. The nanoparticulate thiazepine compositions typically have an effective average particle size of less than about 2000 nm.

### BACKGROUND

### A. Thiazepine Compounds

Thiazepines are compounds having a 7-member heterocyclic ring that includes a nitrogen atom and a sulfur atom. The heterocyclic ring may be partially or completely saturated. Thiazepine compounds have been shown to be useful in treating and preventing a variety of diseases and disorders. For example, thiazepine compounds, methods of their use and synthesis are described in U.S. Pat. Nos. 7,015,212 for “Thiazepine inhibitors of HIV-1 integrase”; 6,235,922 for “Processes and intermediates for preparing benzo-fused azepinone and piperidinone compounds useful in the inhibition of ACE and NEP”; 5,877,313 for “Benzo-fused azepinone and piperidinone compounds useful in the inhibition of ACE and NEP”; 5,859,239 for “Mercaptoalkanoylamino and acylmercaptoalkanoylamino benzoxazepines and benzothiazepines”; 5,856,477 for “Azepinones useful as intermediates in the preparation of inhibitors of angiotensin converting enzyme and neutralendopeptidase”; 5,856,476 for “Processes for preparing azepiones useful as intermediates in the preparation of inhibitors of angiotensin converting enzyme and neutral endopeptidase”; 5,723,602 for “Dual action inhibitors containing a pyridazinodiazepine or pyrazolodiazepine lactam ring”; 5,723,457 for “Acylmercaptoalkanoylamino and mercaptoalkanoylamino benzazepines”; 5,654,294 for “Spiro lactam dual action inhibitors”; 5,650,408 for “Thiazolo benzazepine containing dual action inhibitors”; 5,646,276 for “Diazepine containing dual action inhibitors”; 5,635,504 for “Diazepine containing dual action inhibitors”; 5,552,397 for “Substituted azepinone dual inhibitors of angiotensin converting enzyme and neutral exdopeptidase”; 5,128,467 for “Cyclic sulfur-containing compounds”; 5,082,836 for “Compositions and methods of use of cyclic sulfur-containing compounds”; 5,041,435 for “Cyclic sulfur-containing compounds”; and 4,778,790 for “Perhydrothiazepine and perhydroazepine derivatives and their therapeutic use,” all of which are incorporated herein by reference.

Thiazepine compounds, methods of their use and synthesis also are described in U.S. published application Nos. 2006-0063927 for “Processes for preparing quetiapine and salts thereof”; 20050222120 for “Peptides derivatives comprising thiazepine group for the treatment of hyperlipidermic conditions”; 2005-0153936 for “Neutral endopeptidase (NEP) and human soluble endopeptidase (hSEP) inhibitors for prophylaxis and treatment of neuro-degenerative disorders”; 2005-0080072 for “Process for the preparation of a thiazepine derivative”; 2004-0214812 for “4-Substituted or unsubstituted-7-hydro-1,4-thiazepine-7-[bicyclic or tricyclic heteroaryl] substituted-3,6-dicarboxylic acid derivatives as beta-lactamase inhibitors”; 2003-0134849 for “Thiazepinyl hydroxamic acid derivatives as matrix metalloproteinase inhibitors”; 2002-0103404 for “Process for the nuclear chlorination of meta-xylene”; 2002-0049357 for “Process for the nucleochlorination of ortho-xylene”; and 2002-0010169 for “Substituted 1,4-thiazepine and analogs as activators of caspases and inducers of apoptosis and the use thereof,” all of which are incorporated herein by reference in their entireties.

One thiazepine compound, temocapril, is useful for the treatment of hypertension and related diseases. Temocapril acts by inhibiting angiotensin-converting enzyme (ACE), thereby preventing a chemical in the blood, angiotensin I, from being converted into a substance that increases salt and water retention in the body. Increased salt and water retention lead to high blood pressure. Treating high blood pressure is important because the condition puts a burden on the heart and the arteries, which can lead to permanent damage over time. If untreated, high blood pressure increases the risk of heart attacks, heart failure, stroke, and/or kidney failure. Temocapril is actually a prodrug; its pharmacologically active metabolite, temocaprilat, is excreted predominantly in bile.

Temocapril hydrochloride has the chemical name α-{(2S,6R)-6-[(1S)-1-ethoxy-carbonyl-3-phenyl-propyl]amino-5-oxo-2-(2-thienyl)perhydro-1,4-thiazepin-4-yl}acetic acid hydrochloride. The empirical formula of temocapril hydrochloride is C₂₃H₂₈N₂O₅S₂HCl and its molecular weight is 513.08. The structural formula of temocapril HCl is:

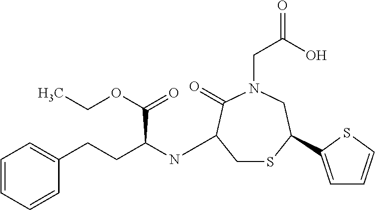

Temocapril hydrochloride is only slightly soluble in water.

Temocapril hydrochloride is offered commercially under the registered trademark ACECOL® by Sankyo Co. Ltd. of Japan. ACECOL® is indicated for the treatment of hypertension and related high blood pressure diseases.

Temocapril is described in, for example, U.S. Pat. Nos. 4,699,905 for “Perhydrothiazepine Derivatives, Their Preparation and Their Therapeutic Use” and 6,610,682 for “Pharmaceutical Compositions and Methods for the Treatment of Arteriosclerosis”, both of which are incorporated herein by reference.

Compositions that include temocapril, either alone or in combination with one or more additional pharmaceutical agents and methods of using and preparing such compositions are described, for example, in U.S. Pat. Nos. 7,022,693 for “Treatment of lipodystrophy”; 6,869,970 for “Crystalline salt forms of valsartan”; 6,787,553 for “Methods for remodeling neuronal and cardiovascular pathways”; 6,767,905 for “Use of angiotensin II receptor antagonists for treating acute myocardial infarction”; 6,747,020 for “Methods of treating heart failure and hypertension using combinations of eplerenone and an angiotensin converting enzyme inhibitor”; 6,653,336 for “Combination of hypertensin converting enzyme inhibitor with a diuretic for treating microcirculation disorders”; 6,610,682 for “Pharmaceutical compositions and methods for the treatment of arteriosclerosis”; 6,599,923 for “Pharmaceutical composition”; 6,465,463 for “Methods of treating and preventing congestive heart failure with hydralazine compounds and isosorbide dinitrate or isosorbide mononitrate”; 6,410,524 for “Combination therapy of angiotensin converting enzyme inhibitor and aldosterone antagonist for reducing morbidity and mortality from cardiovascular disease”; 6,277,869 for “Pharmaceutical composition”; 6,274,605 for “Pharmaceutical composition”; 6,242,432 for “Antithrombotic organic nitrates”; 6,172,089 for “Pharmaceutical composition”; and 6,133,304 for “ACE inhibitor-MMP inhibitor combinations,” all of which are incorporated by reference herein in their entireties.

In addition, compositions that include temocapril, either alone or in combination with one or more additional pharmaceutical agents and methods of using and preparing such compositions also are described, for example, in U.S. published application Nos. 2005-0250748 for “Combination therapy of angiotensin converting enzyme inhibitor and eplerenone for treatment of cardiovascular disease”; 2005-0234043 for “ACE inhibitor-vasopressin antagonist combinations”; 2005-0203168 for “Angiotensin converting enzyme inhibitor use for treatment and prevention of gastrointestinal disorders”; 2004-0167197 for “Compositions, combinations, and methods for treating cardiovascular conditions and other associated conditions”; 2004-0167108 for “Combination therapy of angiotensin converting enzyme inhibitor and aldosterone antagonist for reducing morbidity and mortality from cardiovascular disease”; 2004-0122042 for “Drugs containing chymase inhibitor and ace inhibitors as the active ingredients”; 2004-0087630 for “Combinations”; 2004-0077611 for “Triple therapy of angiotensin converting enzyme inhibitor epoxy-steroidal aldosterone antagonist and diuretic or digoxin for treatment of cardiovascular disease”; 2004-0063719 for “Combination therapy using antihypertensive agents and endothelin antagonists”; 2004-0023840 for “Combination of organic compounds”; 2003-0148960 for “Combination therapy of angiotensin converting enzyme inhibitor and side-effect-reduced amount of aldosterone antagonist for treatment of cardiovascular disease”; 2003-0144213 for “Combination therapy of angiotensin converting enzyme inhibitor, side-effect reduced amount of aldosterone antagonist and diuretic for treatment of cardiovascular disease”; 2003-0103983 for “Ace inhibitor-vasopressin antagonist combinations”; and 2003-0040484 for “Combination therapy of angiotensin converting enzyme inhibitor and aldosterone antagonist for reducing morbidity and mortality from cardiovascular disease,” all of which are incorporated by reference herein in their entireties.

Therefore, thiazepine compounds (e.g. temocapril) can have high therapeutic value in the treatment of hypertension and related diseases. However, because thiazepine compounds (e.g. temocapril) may be practically insoluble in water, significant bioavailability can be problematic. There is a need in the art for a more water soluble and bioavailable formulations of thiazepine compounds. More specifically, there is a need for nanoparticulate temocapril formulations which overcome this and other problems associated with the use of temocapril in the treatment of hypertension and related diseases. The present invention satisfies this need.

### B. Background Regarding Nanoparticulate Active Agent Compositions

Nanoparticulate active agent compositions, first described in U.S. Pat. No. 5,145,684 (“the '684 patent”), are particles consisting of a poorly soluble therapeutic or diagnostic agent having adsorbed onto or associated with the surface thereof a non-crosslinked surface stabilizer. The '684 patent does not describe nanoparticulate compositions of anti-hypertensive compounds such as temocapril.

Methods of making nanoparticulate compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate active agent compositions are also described, for example, in U.S. Pat. Nos. 5,298,262 for “Use of Ionic Cloud Point Modifiers to Prevent Particle Aggregation During Sterilization;” 5,302,401 for “Method to Reduce Particle Size Growth During Lyophilization;” 5,318,767 for “X-Ray Contrast Compositions Useful in Medical Imaging;” 5,326,552 for “Novel Formulation For Nanoparticulate X-Ray Blood Pool Contrast Agents Using High Molecular Weight Non-ionic Surfactants;” 5,328,404 for “Method of X-Ray Imaging Using Iodinated Aromatic Propanedioates;” 5,336,507 for “Use of Charged Phospholipids to Reduce Nanoparticle Aggregation;” 5,340,564 for “Formulations Comprising Olin 10-G to Prevent Particle Aggregation and Increase Stability;” 5,346,702 for “Use of Non-Ionic Cloud Point Modifiers to Minimize Nanoparticulate Aggregation During Sterilization;” 5,349,957 for “Preparation and Magnetic Properties of Very Small Magnetic-Dextran Particles;” 5,352,459 for “Use of Purified Surface Modifiers to Prevent Particle Aggregation During Sterilization;” 5,399,363 and 5,494,683, both for “Surface Modified Anticancer Nanoparticles;” 5,401,492 for “Water Insoluble Non-Magnetic Manganese Particles as Magnetic Resonance Enhancement Agents;” 5,429,824 for “Use of Tyloxapol as a Nanoparticulate Stabilizer;” 5,447,710 for “Method for Making Nanoparticulate X-Ray Blood Pool Contrast Agents Using High Molecular Weight Non-ionic Surfactants;” 5,451,393 for “X-Ray Contrast Compositions Useful in Medical Imaging;” 5,466,440 for “Formulations of Oral Gastrointestinal Diagnostic X-Ray Contrast Agents in Combination with Pharmaceutically Acceptable Clays;” 5,470,583 for “Method of Preparing Nanoparticle Compositions Containing Charged Phospholipids to Reduce Aggregation;” 5,472,683 for “Nanoparticulate Diagnostic Mixed Carbamic Anhydrides as X-Ray Contrast Agents for Blood Pool and Lymphatic System Imaging;” 5,500,204 for “Nanoparticulate Diagnostic Dimers as X-Ray Contrast Agents for Blood Pool and Lymphatic System Imaging;” 5,518,738 for “Nanoparticulate NSAID Formulations;” 5,521,218 for “Nanoparticulate Iododipamide Derivatives for Use as X-Ray Contrast Agents;” 5,525,328 for “Nanoparticulate Diagnostic Diatrizoxy Ester X-Ray Contrast Agents for Blood Pool and Lymphatic System Imaging;” 5,543,133 for “Process of Preparing X-Ray Contrast Compositions Containing Nanoparticles;” 5,552,160 for “Surface Modified NSAID Nanoparticles;” 5,560,931 for “Formulations of Compounds as Nanoparticulate Dispersions in Digestible Oils or Fatty Acids;” 5,565,188 for “Polyalkylene Block Copolymers as Surface Modifiers for Nanoparticles;” 5,569,448 for “Sulfated Non-ionic Block Copolymer Surfactant as Stabilizer Coatings for Nanoparticle Compositions;” 5,571,536 for “Formulations of Compounds as Nanoparticulate Dispersions in Digestible Oils or Fatty Acids;” 5,573,749 for “Nanoparticulate Diagnostic Mixed Carboxylic Anydrides as X-Ray Contrast Agents for Blood Pool and Lymphatic System Imaging;” 5,573,750 for “Diagnostic Imaging X-Ray Contrast Agents;” 5,573,783 for “Redispersible Nanoparticulate Film Matrices With Protective Overcoats;” 5,580,579 for “Site-specific Adhesion Within the GI Tract Using Nanoparticles Stabilized by High Molecular Weight, Linear Poly(ethylene Oxide) Polymers;” 5,585,108 for “Formulations of Oral Gastrointestinal Therapeutic Agents in Combination with Pharmaceutically Acceptable Clays;” 5,587,143 for “Butylene Oxide-Ethylene Oxide Block Copolymers Surfactants as Stabilizer Coatings for Nanoparticulate Compositions;” 5,591,456 for “Milled Naproxen with Hydroxypropyl Cellulose as Dispersion Stabilizer;” 5,593,657 for “Novel Barium Salt Formulations Stabilized by Non-ionic and Anionic Stabilizers;” 5,622,938 for “Sugar Based Surfactant for Nanocrystals;” 5,628,981 for “Improved Formulations of Oral Gastrointestinal Diagnostic X-Ray Contrast Agents and Oral Gastrointestinal Therapeutic Agents;” 5,643,552 for “Nanoparticulate Diagnostic Mixed Carbonic Anhydrides as X-Ray Contrast Agents for Blood Pool and Lymphatic System Imaging;” 5,718,388 for “Continuous Method of Grinding Pharmaceutical Substances;” 5,718,919 for “Nanoparticles Containing the R(−)Enantiomer of Ibuprofen;” 5,747,001 for “Aerosols Containing Beclomethasone Nanoparticle Dispersions;” 5,834,025 for “Reduction of Intravenously Administered Nanoparticulate Formulation Induced Adverse Physiological Reactions;” 6,045,829 “Nanocrystalline Formulations of Human Immunodeficiency Virus (HIV) Protease Inhibitors Using Cellulosic Surface Stabilizers;” 6,068,858 for “Methods of Making Nanocrystalline Formulations of Human Immunodeficiency Virus (HIV) Protease Inhibitors Using Cellulosic Surface Stabilizers;” 6,153,225 for “Injectable Formulations of Nanoparticulate Naproxen;” 6,165,506 for “New Solid Dose Form of Nanoparticulate Naproxen;” 6,221,400 for “Methods of Treating Mammals Using Nanocrystalline Formulations of Human Immunodeficiency Virus (HIV) Protease Inhibitors;” 6,264,922 for “Nebulized Aerosols Containing Nanoparticle Dispersions;” 6,267,989 for “Methods for Preventing Crystal Growth and Particle Aggregation in Nanoparticle Compositions;” 6,270,806 for “Use of PEG-Derivatized Lipids as Surface Stabilizers for Nanoparticulate Compositions;” 6,316,029 for “Rapidly Disintegrating Solid Oral Dosage Form,” 6,375,986 for “Solid Dose Nanoparticulate Compositions Comprising a Synergistic Combination of a Polymeric Surface Stabilizer and Dioctyl Sodium Sulfosuccinate;” 6,428,814 for “Bioadhesive Nanoparticulate Compositions Having Cationic Surface Stabilizers;” 6,431,478 for “Small Scale Mill;” and 6,432,381 for “Methods for Targeting Drug Delivery to the Upper and/or Lower Gastrointestinal Tract,” 6,592,903 for “Nanoparticulate Dispersions Comprising a Synergistic Combination of a Polymeric Surface Stabilizer and Dioctyl Sodium Sulfosuccinate,” 6,582,285 for “Apparatus for sanitary wet milling;” 6,656,504 for “Nanoparticulate Compositions Comprising Amorphous Cyclosporine;” 6,742,734 for “System and Method for Milling Materials;” 6,745,962 for “Small Scale Mill and Method Thereof;” 6,811,767 for “Liquid droplet aerosols of nanoparticulate drugs;” 6,908,626 for “Compositions having a combination of immediate release and controlled release characteristics;” 6,969,529 for “Nanoparticulate compositions comprising copolymers of vinyl pyrrolidone and vinyl acetate as surface stabilizers;” and 6,976,647 for “System and Method for Milling Materials,” all of which are specifically incorporated by reference. In addition, U.S. Patent Publication No. 20020012675 A1, for “Controlled Release Nanoparticulate Compositions;” U.S. Patent Publication No. 20050276974 for “Nanoparticulate Fibrate Formulations;” U.S. Patent Publication No. 20050238725 for “Nanoparticulate compositions having a peptide as a surface stabilizer;” U.S. Patent Publication No. 20050233001 for “Nanoparticulate megestrol formulations;” U.S. Patent Publication No. 20050147664 for “Compositions comprising antibodies and methods of using the same for targeting nanoparticulate active agent delivery;” U.S. Patent Publication No. 20050063913 for “Novel metaxalone compositions;” U.S. Patent Publication No. 20050042177 for “Novel compositions of sildenafil free base;” U.S. Patent Publication No. 20050031691 for “Gel stabilized nanoparticulate active agent compositions;” U.S. Patent Publication No. 20050019412 for “Novel glipizide compositions;” U.S. Patent Publication No. 20050004049 for “Novel griseofulvin compositions;” U.S. Patent Publication No. 20040258758 for “Nanoparticulate topiramate formulations;” U.S. Patent Publication No. 20040258757 for “Liquid dosage compositions of stable nanoparticulate active agents;” U.S. Patent Publication No. 20040229038 for “Nanoparticulate meloxicam formulations;” U.S. Patent Publication No. 20040208833 for “Novel fluticasone formulations;” U.S. Patent Publication No. 20040195413 for “Compositions and method for milling materials;” U.S. Patent Publication No. 20040156895 for “Solid dosage forms comprising pullulan;” U.S. Patent Publication No. U.S. Patent Publication No. U.S. Patent Publication No. 20040156872 for “Novel nimesulide compositions;” U.S. Patent Publication No. 20040141925 for “Novel triamcinolone compositions;” U.S. Patent Publication No. 20040115134 for “Novel nifedipine compositions;” U.S. Patent Publication No. 20040105889 for “Low viscosity liquid dosage forms;” U.S. Patent Publication No. 20040105778 for “Gamma irradiation of solid nanoparticulate active agents;” U.S. Patent Publication No. 20040101566 for “Novel benzoyl peroxide compositions;” U.S. Patent Publication No. 20040057905 for “Nanoparticulate beclomethasone dipropionate compositions;” U.S. Patent Publication No. 20040033267 for “Nanoparticulate compositions of angiogenesis inhibitors;” U.S. Patent Publication No. 20040033202 for “Nanoparticulate sterol formulations and novel sterol combinations;” U.S. Patent Publication No. 20040018242 for “Nanoparticulate nystatin formulations;” U.S. Patent Publication No. 20040015134 for “Drug delivery systems and methods;” U.S. Patent Publication No. 20030232796 for “Nanoparticulate polycosanol formulations & novel polycosanol combinations;” U.S. Patent Publication No. 20030215502 for “Fast dissolving dosage forms having reduced friability;” U.S. Patent Publication No. 20030185869 for “Nanoparticulate compositions having lysozyme as a surface stabilizer;” U.S. Patent Publication No. 20030181411 for “Nanoparticulate compositions of mitogen-activated protein (MAP) kinase inhibitors;” U.S. Patent Publication No. 20030137067 for “Compositions having a combination of immediate release and controlled release characteristics;” U.S. Patent Publication No. 20030108616 for “Nanoparticulate compositions comprising copolymers of vinyl pyrrolidone and vinyl acetate as surface stabilizers;” U.S. Patent Publication No. 20030095928 for “Nanoparticulate insulin;” U.S. Patent Publication No. 20030087308 for “Method for high through put screening using a small scale mill or microfluidics;” U.S. Patent Publication No. 20030023203 for “Drug delivery systems & methods;” U.S. Patent Publication No. 20020179758 for “System and method for milling materials; and U.S. Patent Publication No. 20010053664 for “Apparatus for sanitary wet milling,” describe nanoparticulate active agent compositions and are specifically incorporated by reference.

Amorphous small particle compositions are described, for example, in U.S. Pat. Nos. 4,783,484 for “Particulate Composition and Use Thereof as Antimicrobial Agent;” 4,826,689 for “Method for Making Uniformly Sized Particles from Water-Insoluble Organic Compounds;” 4,997,454 for “Method for Making Uniformly-Sized Particles From Insoluble Compounds;” 5,741,522 for “Ultrasmall, Non-aggregated Porous Particles of Uniform Size for Entrapping Gas Bubbles Within and Methods;” and 5,776,496, for “Ultrasmall Porous Particles for Enhancing Ultrasound Back Scatter.”

The present invention then, relates to a nanoparticulate formulation that may be useful in treating and preventing diseases and disorders that include hypertension, high blood pressure, heart attack, stroke, kidney failure and other high blood pressure-related diseases. More specifically, the present invention relates to a nanoparticulate formulation of a thiazepine compound, such as temocapril, in which the active ingredient is formulated as discreet particles having an effective average particle size of less than about 2000 nm. Typically, the discreet particles include one or more surface stabilizers. The active ingredient may include the thiazepine compound or a salt or derivative thereof. For example, the active ingredient may include temocapril or a salt or derivative thereof. The nanoparticulate temocapril active ingredient may be formulated as a pharmaceutically acceptable composition for the treatment of hypertension and related diseases.

### SUMMARY

Disclosed are nanoparticulate compositions comprising a compound that may be useful for treating or preventing diseases and disorders such as hypertension. For example, the compound may include a thiazepine compound having anti-hypertensive pharmaceutical properties, such as temocapril, or a salt or derivative thereof. The compositions may include nanoparticulate temocapril particles, and at least one surface stabilizer adsorbed on or associated with the surface of the temocapril particles. The nanoparticulate temocapril particles may have an effective average particle size of less than about 2000 nm.

A preferred dosage form of the invention is a solid dosage form, although any pharmaceutically acceptable dosage form can be utilized.

Another aspect of the invention is directed to pharmaceutical compositions comprising particles of a nanoparticulate thiazepine compound or a salt or derivative thereof, such as temocapril or temocarpil hydrochloride, at least one surface stabilizer, a pharmaceutically acceptable carrier, and any desired excipients.

Another embodiment of the invention is directed to nanoparticulate antihypertensive compositions comprising one or more compounds useful in the treatment or prevention of diseases such as hypertension. For example, the nanoparticulate compositions may include nanoparticulate temocapril and one or more compounds useful in the treatment of hypertension.

This invention further discloses a method of making the inventive nanoparticulate anti-hypertensive compositions. Such a method comprises contacting the nanoparticulate thiazepine compound, such as temocapril or a salt or derivative thereof, with at least one surface stabilizer for a time and under conditions sufficient to provide a stabilized nanoparticulate thiazepine composition.

The present invention is also directed to methods of treatment including but not limited to, the treatment of hypertension and related diseases, using the novel nanoparticulate thiazepine compositions disclosed herein. Such methods may comprise administering to a subject a therapeutically effective amount of a nanoparticulate thiazepine compound, such as temocapril, or a salt or derivative thereof. Other methods of treatment using the nanoparticulate compositions of the invention are known to those of skill in the art.

### DETAILED DESCRIPTION OF THE INVENTION

### I. Nanoparticulate Compositions of Thiazepine Compounds

The present invention is directed to nanoparticulate compositions comprising particles of a thiazepine compound having anti-hypertensive pharmaceutical properties, such as temocapril, or a salt or derivative thereof and preferably at least one surface stabilizer. The surface stabilizer can be adsorbed on or associated with the surface of the drug particles. The thiazepine compound, such as temocapril or a salt or derivative thereof, particles may have an effective average particle size of less than about 2000 nm.

Advantages of nanoparticulate thiazepine formulations as compared to prior conventional, non-nanoparticulate or microcrystalline dosage forms of the same thiazepine include, but are not limited to: (1) smaller tablet or other solid dosage form size; (2) smaller doses of drug required to obtain the same pharmacological effect; (3) increased bioavailability; (4) improved pharmacokinetic profiles; (5) an increased rate of dissolution; and (6) the thiazepine compositions can be used in conjunction with other active agents useful in the treatment of hypertension and related diseases.

Also disclosed are compositions that include a nanoparticulate thiazepine compound, such as temocapril, or a salt or derivative thereof, together with one or more non-toxic physiologically acceptable carriers, adjuvants, or vehicles, collectively referred to as carriers. The compositions can be formulated for parental injection (e.g., intravenous, intramuscular, or subcutaneous), oral administration in solid, liquid, or aerosol form, vaginal, nasal, rectal, otically, ocular, local (powders, ointments, or drops), buccal, intracistemal, intraperitoneal, or topical administrations, and the like.

A preferred dosage form of the invention is a solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. A solid dose tablet formulation is preferred. Solid dosage forms also may be used to prepare suspensions, dispersions, or emulsions.

The present invention is described herein using several definitions, as set forth below and throughout the application.

The term “effective average particle size,” as used herein, means that at least about 50% of the nanoparticulate thiazepine compound particles, such as temocapril, have a size of less than about 2000 nm, by weight or by other suitable measurement technique (e.g., such as by volume, number, etc.), when measured by, for example, sedimentation flow fractionation, photon correlation spectroscopy, light scattering, disk centrifugation, and other techniques known to those of skill in the art.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to stable thiazepine compound particles (e.g., stable nanoparticles of temocapril), “stable” means that the particles do not appreciably flocculate or agglomerate due to interparticle attractive forces or otherwise increase in particle size. “Stable” connotes, but is not limited to one or more of the following parameters: (1) the particles do not appreciably flocculate or agglomerate due to interparticle attractive forces or otherwise significantly increase in particle size over time; (2) the physical structure of the particles is not altered over time, such as by conversion from an amorphous phase to a crystalline phase; (3) the particles are chemically stable; and/or (4) where the thiazepine compound has not been subject to a heating step at or above the melting point of the thiazepine compound particles in the preparation of the nanoparticles of the present invention.

The term “conventional” or “non-nanoparticulate active agent” shall mean an active agent which is solubilized or which has an effective average particle size of greater than about 2000 nm. Nanoparticulate active agents as defined herein have an effective average particle size of less than about 2000 nm.

The phrase “poorly water soluble drugs” as used herein refers to those drugs that have a solubility in water of less than about 30 mg/ml, less than about 20 mg/ml, less than about 10 mg/ml, or less than about 1 mg/ml.

As used herein, the phrase “therapeutically effective amount” shall mean that drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

A. Preferred Characteristics of Nanoparticulate Thiazepine Compositions of the Invention

1. Increased Bioavailability

The nanoparticulate thiazepine compositions may include nanoparticulate temocapril. The nanoparticulate temocapril, or a salt or derivative thereof, formulations of the invention are proposed to exhibit increased bioavailability, and require smaller doses as compared to prior conventional temocapril formulations.

For example, a nanoparticulate formulation of temocapril may show reduced particle size and increased bioavailability as compared to a conventional formulation of temocapril. The increased bioavailability is significant because it means that a nanoparticulate temocapril dosage form exhibits significantly greater drug absorption.

2. Improved Pharmacokinetic Profiles

The invention also provides nanoparticulate thiazepine compositions having a desirable pharmacokinetic profile when administered to mammalian subjects. The desirable pharmacokinetic profile of the compositions comprising a thiazepine, such as temocapril, includes but is not limited to: (1) a Cₘₐₓ for a thiazepine, such as temocapril, when assayed in the plasma of a mammalian subject following administration, that is preferably greater than the Cₘₐₓ for a non-nanoparticulate formulation of the same thiazepine, administered at the same dosage; and/or (2) an AUC for a thiazepine, such as temocapril, when assayed in the plasma of a mammalian subject following administration, that is preferably greater than the AUC for a non-nanoparticulate formulation of the same thiazepine, administered at the same dosage; and/or (3) a Tₘₐₓ for a thiazepine, such as temocapril, when assayed in the plasma of a mammalian subject following administration, that is preferably less than the Tₘₐₓ for a non-nanoparticulate formulation of the same thiazepine, administered at the same dosage. The desirable pharmacokinetic profile, as used herein, is the pharmacokinetic profile measured after the initial dose of a thiazepine, such as temocapril.

In one embodiment, a composition comprising a nanoparticulate thiazepine, such as temocapril, exhibits in comparative pharmacokinetic testing with a non-nanoparticulate formulation of the same thiazepine, administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate thiazepine formulation.

In another embodiment, the composition comprising a nanoparticulate thiazepine, such as temocapril, exhibits in comparative pharmacokinetic testing with a non-nanoparticulate formulation of the same thiazepine, administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate thiazepine formulation.

In yet another embodiment, the composition comprising a nanoparticulate thiazepine, such as temocapril, exhibits in comparative pharmacokinetic testing with a non-nanoparticulate formulation of the same thiazepine, administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate thiazepine formulation.

In one embodiment of the invention, the Tₘₐₓ of the thiazepine, such as temocapril, when assayed in the plasma of the mammalian subject, is less than about 6 to about 8 hours. In other embodiments of the invention, the Tₘₐₓ of the thiazepine is less than about 6 hours, less than about 5 hours, less than about 4 hours, less than about 3 hours, less than about 2 hours, less than about 1 hour, or less than about 30 minutes after administration.

The desirable pharmacokinetic profile, as used herein, is the pharmacokinetic profile measured after the initial dose of a thiazepine, such as temocapril. The compositions can be formulated in any way as described herein and as known to those of skill in the art.

3. The Pharmacokinetic Profiles of the Thiazepine Compositions of the Invention are not Affected by the Fed or Fasted State of the Subject Ingesting the Compositions

The invention encompasses thiazepine, such as temocapril, compositions where the pharmacokinetic profile of the thiazepine, such as temocapril, is not substantially affected by the fed or fasted state of a subject ingesting the composition. This means that there is no substantial difference in the quantity of drug absorbed or the rate of drug absorption when the nanoparticulate thiazepine, such as temocapril, compositions are administered in the fed versus the fasted state.

The difference in absorption of the thiazepine, such as temocapril or a salt or derivative thereof, compositions of the invention, when administered in the fed versus the fasted state, preferably is less than about 35%, less than about 30%, less than about 25%, less than about 20%, less than about 15%, less than about 10%, less than about 5%, or less than about 3%.

4. Bioequivalency of α₄ Integrin Antagonist Compositions of the Invention When Administered in the Fed Versus the Fasted State

The invention also encompasses a nanoparticulate thiazepine, such as temocapril or a salt or derivative thereof, composition in which administration of the composition to a subject in a fasted state is bioequivalent to administration of the composition to a subject in a fed state.

The difference in absorption (AUC) or Cₘₐₓ of a thiazepine, such as temocapril, compositions of the invention, when administered in the fed versus the fasted state, preferably is less than about 60%, less than about 55%, less than about 50%, less than about 45%, less than about 40%, less than about 35%, less than about 30%, less than about 25%, less than about 20%, less than about 15%, less than about 10%, less than about 5%, or less than about 3%.

In one embodiment of the invention, the invention encompasses compositions comprising a nanoparticulate thiazepine, such as temocapril, wherein administration of the composition to a subject in a fasted state is bioequivalent to administration of the composition to a subject in a fed state, in particular as defined by Cₘₐₓ and AUC guidelines given by the U.S. Food and Drug Administration and the corresponding European regulatory agency (EMEA). Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must between 0.70 to 1.43.

5. Dissolution Profiles of the Thiazepine Compositions of the Invention

The nanoparticulate thiazepine, such as temocapril or a salt or derivative thereof, compositions of the invention are proposed to have unexpectedly dramatic dissolution profiles. Rapid dissolution of an administered active agent is preferable, as faster dissolution generally leads to faster onset of action and greater bioavailability. To improve the dissolution profile and bioavailability of a thiazepine such as temocapril it would be useful to increase the drug's dissolution so that it could attain a level close to 100%.

In one embodiment of the invention, the thiazepine, such as temocapril or a salt or derivative thereof, compositions of the invention have a dissolution profile in which at least about 30% of the composition is dissolved within 5 minutes, at least about 70% is dissolved within 10 minutes, at least about 90% is dissolved within 15 minutes, and at least about 95% is dissolved within about 20, about 25, about 30 or about 45 minutes.

In another embodiment of the invention, the thiazepine, such as temocapril or a salt or derivative thereof, compositions of the invention have a dissolution profile in which within about 5 minutes at least about 20% of the composition is dissolved. In other embodiments of the invention, at least about 30% or at least about 40% of the thiazepine, such as temocapril or a salt or derivative thereof, composition is dissolved within about 5 minutes. In yet other embodiments of the invention, at least 40%, at least about 50%, at least about 60%, at least about 70%, or at least about 80% of the thiazepine, such as temocapril or a salt or derivative thereof, composition is dissolved within about 10 minutes. Finally, in another embodiment of the invention, at least about 70%, at least about 80%, at least about 90%, or at least about 100% of the thiazepine, such as temocapril or a salt or derivative thereof, composition is dissolved within 20 minutes.

Dissolution is preferably measured in a medium which is discriminating. Such a dissolution medium will produce two very different dissolution curves for two products having very different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

6. Redispersability of the Thiazepine Compositions of the Invention

An additional feature of the thiazepine, such as temocapril or a salt or derivative thereof, compositions of the invention is that the compositions redisperse such that the effective average particle size of the redispersed thiazepine particles is less than about 2 microns. This is significant, as if upon administration the thiazepine compositions of the invention did not redisperse to a substantially nanoparticulate size, then the dosage form may lose the benefits afforded by formulating the temocapril into a nanoparticulate size.

This is because nanoparticulate active agent compositions benefit from the small particle size of the active agent; if the active agent does not disperse into the small particle sizes upon administration, them “clumps” or agglomerated active agent particles are formed, owing to the extremely high surface free energy of the nanoparticulate system and the thermodynamic driving force to achieve an overall reduction in free energy. With the formulation of such agglomerated particles, the bioavailability of the dosage form my fall well below that observed with the liquid dispersion form of the nanoparticulate active agent.

In other embodiments of the invention, the redispersed thiazepine, such as temocapril or a salt or derivative thereof, particles of the invention have an effective average particle size of less than about less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

Moreover, the nanoparticulate thiazepine, such as temocapril or a salt or derivative thereof, compositions of the invention exhibit dramatic redispersion of the nanoparticulate thiazepine particles upon administration to a mammal, such as a human or animal, as demonstrated by reconstitution/redispersion in a biorelevant aqueous media such that the effective average particle size of the redispersed thiazepine particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 M, and NaCl solutions, ranging in concentration from about 0.001 to about 0.1 M, and mixtures thereof. For example, electrolyte solutions can be, but are not limited to, about 0.1 M HCl or less, about 0.01 M HCl or less, about 0.001 M HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 M HCl, 0.01 M HCl, and 0.1 M HCl correspond to pH 3, pH 2, and pH 1, respectively. Thus, a 0.01 M HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed thiazepine, such as temocapril or a salt or derivative thereof, particles of the invention (redispersed in an aqueous, biorelevant, or any other suitable media) have an effective average particle size of less than about less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. Such methods suitable for measuring effective average particle size are known to a person of ordinary skill in the art.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

7. Thiazepine Compositions Used in Conjunction with Other Active Agents

The thiazepine, such as temocapril or a salt or derivative thereof, compositions of the invention can additionally comprise one or more compounds useful in the treatment of hypertension and related diseases, or the thiazepine, such as temocapril or a salt or derivative thereof, compositions can be administered in conjunction with such a compound. Such compounds include, but are not limited to diuretics (e.g., amiloride, bendroflumethiazide, benzthiazide, bumetanide, chlorothiazide, chlorthalidone, fusosemide, hydrochlorothiazide, hydroflumethiazide, indapamide, methyclothiazide, metolazone, polythiazide, spironolactone, torsemide, triamterene, and trichlomethiazide), beta blockers (e.g., acebutalol, atenolol, betaxolol, bisoprolol, carteolol, esmolol, metoprolol, nadolol, penbutolol, pindolol, propranolol, sotalol, and timolol), other ACE inhibitors (e.g., benazepril, captopril, cilazapril, enalapril, fosinopril, lisinopril, moexipril, perindopril, quinapril, ramipril, and trandolapril), calcium channel blockers (e.g., amlodipine, bepridil, diltiazam, felodipine, flunarizine, isradipine, nicardinpine, nifedipine, nimodipine, nisoldipine, and verapamil), alpha blockers (e.g., doxazosin, prazosin, and terazosin), alpha-beta blockers (e.g., labetalol, and carvedilol), angiotensin antagonists (e.g., losartan and valsartan), nervous system inhibitors (e.g., guanabenz, guanadrel, guanethidine, guanfacine, methyldopa, and reserpine), and vasodilators (e.g. hydralazine and minoxidil).

B. Nanoparticulate Temocapril Compositions

The invention provides compositions comprising thiazepine, such as temocapril or a salt or derivative thereof, particles and at least one surface stabilizer. The surface stabilizers preferably are adsorbed on or associated with the surface of the thiazepine, such as temocapril or a salt or derivative thereof, particles. Surface stabilizers especially useful herein preferably physically adhere on, or associate with, the surface of the nanoparticulate thiazepine particles, but do not chemically react with the thiazepine, such as temocapril or a salt or derivative thereof, particles or itself. Individually adsorbed molecules of the surface stabilizer are essentially free of intermolecular cross-linkages.

The invention also includes thiazepine, such as temocapril or a salt or derivative thereof, compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracistemal, intraperitoneal, or topical administration, and the like.

1. Thiazepine Compounds

Thiazepine compounds present in the compositions of the invention have anti-hypertensive pharmaceutical properties and can be in a crystalline phase, semi-crystalline phase, amorphous phase, semi-amorphous phase, or a combination thereof.

Thiazepine compounds include a 7-member heterocyclic ring that includes a nitrogen atom and a sulfur atom. Optionally, the thiazepine is saturated at one or more positions. The thiazepine compound for the compositions disclosed herein may include a “perhydrothiazepine” which is completely saturated. Suitable perhydrothiazepine compounds for the compositions disclosed herein may include 1,4-thiazepines having the formula

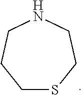

In some embodiments, the nanoparticulate thiazepine formulations disclosed herein include an oxo-1,4-thiazepine, such as 5-oxo-thiazepine having the formula.

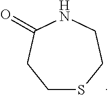

A 5-oxo-thiazepine suitable for the compositions disclosed herein may include a compound having the formula:

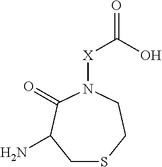

where X is C₁₋₆-alkylene.

In further embodiments, a thiazepine compound suitable for the compositions disclosed herein may have a formula:

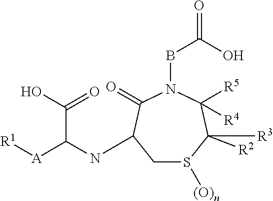

where: R¹ represents an optionally substituted alkyl, cycloalkyl, aryl, partially hydrogenated aryl or heterocyclic group; R², R³, R⁴ and R⁵ represent hydrogen or an optionally substituted alkyl, cycloalkyl, aralkyl, aryl, heterocyclic or heterocyclic-alkyl group or any adjacent pair thereof form a cyclic structure, at least one not being hydrogen; A represents a bond, or a methylene, ethylene, oxymethyl or thiomethyl group; B represents an alkylene, alkylidene, cycloalkylene or cycloalkylidene group; and n is 0, 1 or 2) and salts and esters thereof. Preferably, the compound used to prepare the nanoparticulate formulations disclosed herein is temocapril or a salt thereof (e.g., temocapril hydrochloride).

2. Surface Stabilizers

Combinations of more than one surface stabilizers can be used in the invention. Useful surface stabilizers which can be employed in the invention include, but are not limited to, known organic and inorganic pharmaceutical excipients. Such excipients include various polymers, low molecular weight oligomers, natural products, and surfactants. Surface stabilizers include nonionic and ionic (e.g., anionic, cationic, or zwitterionic) compounds or surfactants.

Representative examples of surface stabilizers include hydroxypropyl methylcellulose (now known as hypromellose), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-lOG® or Surfactant 10-G (Olin Chemicals, Stamford, Conn.); Crodestas SL-40® (Croda, Inc.); and SA9OHCO, which is C₁₈H₃₇CH₂(CON(CH₃)—CH₂(CHOH)₄(CH₂0H)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, such as Plasdone S630, and the like.

If desirable, the nanoparticulate temocapril compositions can be formulated to be phospholipids-free. A composition is phospholipids-free where the composition includes less than about 0.1% phospholipids (w/w).

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅-dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants Physical Chemistry (Marcel Dekker, 1991); and J. Richmond, Cationic Surfactants Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:

(i) none of R₁-R₄ are CH₃;

(ii) one of R₁-R₄ is CH₃;

(iii) three of R₁-R₄ are CH₃;

(iv) all of R₁-R₄ are CH₃;

(v) two of R₁-R₄ are CH₃, one of R₁-R₄ is C₆H₅CH₂, and one of R₁-R₄ is an alkyl chain of seven carbon atoms or less;

(vi) two of R₁-R₄ are CH₃, one of R₁-R₄ is C₆H₅CH₂, and one of R₁-R₄ is an alkyl chain of nineteen carbon atoms or more;

(vii) two of R₁-R₄ are CH₃ and one of R₁-R₄ is the group C₆H₅(CH₂)ₙ, where n>1;

(viii) two of R₁-R₄ are CH₃, one of R₁-R₄ is C₆H₅CH₂, and one of R₁-R₄ comprises at least one heteroatom;

(ix) two of R₁-R₄ are CH₃, one of R₁-R₄ is C₆H₅CH₂, and one of R₁-R₄ comprises at least one halogen;

(x) two of R₁-R₄ are CH₃, one of R₁-R₄ is C₆H₅CH₂, and one of R₁-R₄ comprises at least one cyclic fragment;

(xi) two of R₁-R₄ are CH₃ and one of R₁-R₄ is a phenyl ring; or

(xii) two of R₁-R₄ are CH₃ and two of R₁-R₄ are purely aliphatic fragments.

Such compounds include, but are not limited to, benzalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The surface stabilizers are commercially available and/or can be prepared by techniques known in the art. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

The temocapril and surface stabilizer may be present in the pharmaceutical compositions disclosed herein at any suitable ratio (w/w) For example, in some embodiments the pharmaceutical compositions include temocapril and the surface stabilizer at a ratio of about 20:1, 15:1, 10:1, 8:1, 7:1, 6:1, 5:1, 4:1, 3:1, 2:1 (w/w), or any range defined by said ratios (for example, but not limited to about 20:1-2:1, about 10:1-4:1, and about 8:1-5:1).

3. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, sucralose, and acesulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

4. Nanoparticulate Thiazepine Particle Size

The anti-hypertensive compositions of the invention comprise a nanoparticulate thiazepine, such as temocapril or a salt or derivative thereof, in the form of stabilized particles which have an effective average particle size of less than about 2000 nm (i.e., 2 microns), less than about 1900 nm, less than about 1800 nm, less than about 1700 mm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

By “an effective average particle size of less than about 2000 nm” it is meant that at least 50% of the thiazepine, such as temocapril or a salt or derivative thereof, particles have a particle size of less than the effective average, by weight (or by other suitable measurement technique, such as by volume, number, etc.), i.e., less than about 2000 nm, 1900 nm, 1800 nm, etc., when measured by the above-noted techniques. In other embodiments of the invention, at least about 60%, at least about 70%, at least about 80%, at least about 90%, at least about 95%, or at least about 99% of the thiazepine, such as temocapril or a salt or derivative thereof, particles have a particle size of less than the effective average, i.e., less than about 2000 nm, 1900 nm, 1800 nm, 1700 nm, etc.

In the present invention, the value for D50 of a nanoparticulate thiazepine, such as temocapril or a salt or derivative thereof, composition is the particle size below which 50% of the thiazepine particles fall, by weight (or by other suitable measurement technique, such as by volume, number, etc.). Similarly, D90 is the particle size below which 90% of the thiazepine particles fall, by weight (or by other suitable measurement technique, such as by volume, number, etc.).

5. Concentration of Temocapril and Surface Stabilizers

The relative amounts of a thiazepine, such as temocapril or a salt or derivative thereof, and one or more surface stabilizers can vary widely. The optimal amount of the individual components can depend, for example, upon the particular thiazepine selected, the hydrophilic lipophilic balance (HLB), melting point, and the surface tension of water solutions of the stabilizer, etc.

The concentration of the thiazepine, such as temocapril or a salt or derivative thereof, can vary from about 99.5% to about 0.001%, from about 95% to about 0.1%, or from about 90% to about 0.5%, by weight, based on the total combined weight of the thiazepine and at least one surface stabilizer, not including other excipients.

The concentration of the at least one surface stabilizer can vary from about 0.5% to about 99.999%, from about 5.0% to about 99.9%, or from about 10% to about 99.5%, by weight, based on the total combined dry weight of the thiazepine and at least one surface stabilizer, not including other excipients.

6. Exemplary Nanoparticulate Temocapril Hydrochloride Tablet Formulations

Several exemplary temocapril hydrochloride tablet formulations are given below. These examples are not intended to limit the claims in any respect, but rather to provide exemplary tablet formulations of temocapril hydrochloride which can be utilized in the methods of the invention. Such exemplary tablets can also comprise a coating agent.

Exemplary Nanoparticulate Temocapril Hydrochloride
Tablet Formulation #1

| Component | g/Kg |
|---|---|
| Temocapril Hydrochloride | about 50 to about 500 |
| Hypromellose, USP | about 10 to about 70 |
| Docusate Sodium, USP | about 1 to about 10 |
| Sucrose, NF | about 100 to about 500 |
| Sodium Lauryl Sulfate, NF | about 1 to about 40 |
| Lactose Monohydrate, NF | about 50 to about 400 |
| Silicified Microcrystalline Cellulose | about 50 to about 300 |
| Crospovidone, NF | about 20 to about 300 |
| Magnesium Stearate, NF | about 0.5 to about 5 |

Exemplary Nanoparticulate Temocapril Hydrochloride
Tablet Formulation #2

| Component | g/Kg |
|---|---|
| Temocapril Hydrochloride | about 100 to about 300 |
| Hypromellose, USP | about 30 to about 50 |
| Docusate Sodium, USP | about 0.5 to about 10 |
| Sucrose, NF | about 100 to about 300 |
| Sodium Lauryl Sulfate, NF | about 1 to about 30 |
| Lactose Monohydrate, NF | about 100 to about 300 |
| Silicified Microcrystalline Cellulose | about 50 to about 200 |
| Crospovidone, NF | about 50 to about 200 |
| Magnesium Stearate, NF | about 0.5 to about 5 |

Exemplary Nanoparticulate Temocapril Hydrochloride
Tablet Formulation #3

| Component | g/Kg |
|---|---|
| Temocapril Hydrochloride | about 200 to about 225 |
| Hypromellose, USP | about 42 to about 46 |
| Docusate Sodium, USP | about 2 to about 6 |
| Sucrose, NF | about 200 to about 225 |
| Sodium Lauryl Sulfate, NF | about 12 to about 18 |
| Lactose Monohydrate, NF | about 200 to about 205 |
| Silicified Microcrystalline Cellulose | about 130 to about 135 |
| Crospovidone, NF | about 112 to about 118 |
| Magnesium Stearate, NF | about 0.5 to about 3 |

Exemplary Nanoparticulate Temocapril Hydrochloride
Tablet Formulation #4

| Component | g/Kg |
|---|---|
| Temocapril Hydrochloride | about 119 to about 224 |
| Hypromellose, USP | about 42 to about 46 |
| Docusate Sodium, USP | about 2 to about 6 |
| Sucrose, NF | about 119 to about 224 |
| Sodium Lauryl Sulfate, NF | about 12 to about 18 |
| Lactose Monohydrate, NF | about 119 to about 224 |
| Silicified Microcrystalline Cellulose | about 129 to about 134 |
| Crospovidone, NF | about 112 to about 118 |
| Magnesium Stearate, NF | about 0.5 to about 3 |

C. Methods of Making Nanoparticulate Thiazepine Compositions

The nanoparticulate thiazepine, such as temocapril or a salt or derivative thereof, compositions can be made using, for example, milling, homogenization, precipitation, freezing, or template emulsion techniques. Exemplary methods of making nanoparticulate active agent compositions are described in the '684 patent. Methods of making nanoparticulate active agent compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

The resultant nanoparticulate thiazepine, such as temocapril or a salt or derivative thereof, compositions or dispersions can be utilized in solid or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc.

1. Milling to Obtain Nanoparticulate Thiazepine Dispersions

Milling a thiazepine, such as temocapril or a salt or derivative thereof to obtain a nanoparticulate thizaepine dispersion comprises dispersing the thiazepine particles in a liquid dispersion medium in which the thiazepine is poorly soluble, followed by applying mechanical means in the presence of grinding media to reduce the particle size of the thiazepine to the desired effective average particle size. The dispersion medium can be, for example, water, safflower oil, ethanol, t-butanol, glycerin, polyethylene glycol (PEG), hexane, or glycol. A preferred dispersion medium is water.

The thiazepine, such as temocapril or a salt or derivative thereof particles can be reduced in size in the presence of at least one surface stabilizer. Alternatively, thizepine particles can be contacted with one or more surface stabilizers after attrition. Other compounds, such as a diluent, can be added to the thiazepine/surface stabilizer composition during the particle size reduction process. Dispersions can be manufactured continuously or in a batch mode.

2. Precipitation to Obtain Nanoparticulate Thiazepine Compositions

Another method of forming the desired nanoparticulate thiazepine, such as temocapril or a salt or derivative thereof, composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving a thiazepine, such as temocapril or a salt or derivative thereof, in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

3. Homogenization to Obtain Nanoparticulate Thiazepine Compositions

Exemplary homogenization methods of preparing active agent nanoparticulate compositions are described in U.S. Pat. No. 5,510,118, for “Process of Preparing Therapeutic Compositions Containing Nanoparticles.” Such a method comprises dispersing particles of a thiazepine, such as temocapril or a salt or derivative thereof, in a liquid dispersion medium, followed by subjecting the dispersion to homogenization to reduce the particle size of the thiazepine, such as temocapril or a salt or derivative thereof, to the desired effective average particle size. The thiazepine particles can be reduced in size in the presence of at least one surface stabilizer. Alternatively, the thiazepine particles can be contacted with one or more surface stabilizers either before or after attrition. Other compounds, such as a diluent, can be added to the thiazepine/surface stabilizer composition either before, during, or after the size reduction process. Dispersions can be manufactured continuously or in a batch mode.

4. Cryogenic Methodologies to Obtain Nanoparticulate Thiazepine Compositions

Another method of forming the desired nanoparticulate thiazepine, such as temocapril or a salt or derivative thereof, compositions is by spray freezing into liquid (SFL). This technology comprises an organic or organoaqueous solution of a thiazepine with surface stabilizers, which is injected into a cryogenic liquid, such as liquid nitrogen. The droplets of the thiazepine, such as temocapril or a salt or derivative thereof, solution freeze at a rate sufficient to minimize crystallization and particle growth, thus formulating nanostructured thiazepine particles. Depending on the choice of solvent system and processing conditions, the nanoparticulate thiazepine particles can have varying particle morphology. In the isolation step, the nitrogen and solvent are removed under conditions that avoid agglomeration or ripening of the thiazepine particles.

As a complementary technology to SFL, ultra rapid freezing (URF) may also be used to created equivalent nanostructured temocapril particles with greatly enhanced surface area. URF comprises an organic or organoaqueous solution of temocapril with stabilizers onto a cryogenic substrate.

5. Emulsion Methodologies to Obtain Nanoparticulate Thiazepine Compositions

Another method of forming the desired nanoparticulate thiazepine, such as temocapril or a salt or derivative thereof, composition is by template emulsion. Template emulsion creates nanostructured thiazepine particles with controlled particle size distribution and rapid dissolution performance. The method comprises an oil-in-water emulsion that is prepared, then swelled with a non-aqueous solution comprising the thiazepine, such as temocapril or a salt or derivative thereof, and surface stabilizers. The particle size distribution of the thiazepine particles is a direct result of the size of the emulsion droplets prior to loading with the thiazepine a property which can be controlled and optimized in this process. Furthermore, through selected use of solvents and stabilizers, emulsion stability is achieved with no or suppressed Ostwald ripening. Subsequently, the solvent and water are removed, and the stabilized nanostructured thiazepine, such as temocapril or a salt or derivative thereof, particles are recovered. Various thiazepine particles morphologies can be achieved by appropriate control of processing conditions.

D. Methods of Using the Nanoparticulate Thiazepine Compositions of the Invention

The invention provides a method of increasing bioavailability of a thiazepine, such as temocapril or a salt or derivative thereof, in a subject. Such a method comprises orally administering to a subject an effective amount of a composition comprising a thiazepine, such as temocapril or a salt or derivative thereof. In one embodiment of the invention, the thiazepine composition, in accordance with standard pharmacokinetic practice, may produce a maximum blood plasma concentration profile in less than about 6 hours, less than about 5 hours, less than about 4 hours, less than about 3 hours, less than about 2 hours, less than about 1 hour, or less than about 30 minutes after the initial dose of the composition.

The compositions of the invention are useful in the treatment of hypertension and related diseases. Diseases related to hypertension include, but are not limited to, ischemic heart disease, stroke, peripheral artery disease, hypertensive heart disease, and renal failure.

The thiazepine, such as temocapril or a salt or derivative thereof, compositions of the invention can be administered to a subject via any conventional means including, but not limited to, orally, rectally, ocularly, parenterally (e.g., intravenous, intramuscular, or subcutaneous), intracistemally, pulmonary, intravaginally, intraperitoneally, locally (e.g., powders, ointments or drops), or as a buccal or nasal spray. As used herein, the term “subject” is used to mean an animal, preferably a mammal, including a human or non-human. The terms patient and subject may be used interchangeably.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate thiazepine, such as temocapril or a salt or derivative thereof, compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms of a nanoparticulate thiazepine, such as temocapril or a salt or derivative thereof, for oral administration include, but are not limited to, capsules, tablets, pills, powders, and granules. In such solid dosage forms, the thiazepine is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms of a nanoparticulate thiazepine, such as temocapril or a salt or derivative thereof, for oral administration include pharmaceutically acceptable emulsions, solutions, suspensions, syrups, and elixirs. In addition to a thiazepine, such as temocapril or a salt or derivative thereof, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

In one illustrated embodiment, the pharmaceutical compositions disclosed herein include a stabilized nanoparticulate thiazepine compound (e.g., nanoparticulate temocapril and a surface stabilizer) and a cellulose-based binder and/or disintegrant (e.g., povidone or crospovidone). Optionally, the pharmaceutical compositions further include a sugar (e.g., sucrose) and/or a sugar alcohol (e.g., mannitol).

One of ordinary skill will appreciate that effective amounts of a thiazepine, such as temocapril or a salt or derivative thereof, can be determined empirically and can be employed in pure form or, where such forms exist, in pharmaceutically acceptable salt, ester, or prodrug form. Actual dosage levels of a thiazepine, such as temocapril or a salt or derivative thereof, in the nanoparticulate compositions of the invention may be varied to obtain an amount of a thiazepine, such as temocapril or a salt or derivative thereof, that is effective to obtain a desired therapeutic response for a particular composition and method of administration. The selected dosage level therefore depends upon the desired therapeutic effect, the route of administration, the potency of the administered thiazepine, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

The following prophetic example is given to illustrate the present invention. It should be understood, however, that the spirit and scope of the invention is not to be limited to the specific conditions or details described in this example but should only be limited by the scope of the claims that follow. All references identified herein, including U.S. patents, are hereby expressly incorporated by reference.

### Example 1

The purpose of this example was to prepare a composition comprising a nanoparticulate thiazepine, such as temocapril or a salt or derivative thereof.

An aqueous dispersion of 5% (w/w) temocapril hydrochloride, combined with one or more surface stabilizers, such as hydroxypropyl cellulose (HPC-SL) and dioctylsulfosuccinate (DOSS), could be milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical Co.) (89% media load). In an exemplary process, the mixture could be milled at a speed of 2500 rpms for 60 minutes.

Following milling, the particle size of the milled temocapril hydrochloride particles can be measured, in deionized distilled water, using a Horiba LA 910 particle size analyzer. The initial mean and/or D50 milled temocapril hydrochloride particle size is expected to be less than 2000 nm.

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

1. A stable nanoparticulate anti-hypertensive pharmaceutical composition comprising:
(a) particles of a thiazepine compound having anti-hypertensive pharmaceutical properties and having an effective average particle size of less than 2000 nm; and
(b) at least one surface stabilizer.

2. The composition of claim 1, wherein the thiazepine is temocapril or a salt thereof.

3. The composition of claim 2 wherein the salt is the hydrochloride salt.

4. The composition of claim 1, wherein the thiazepine particle is in a crystalline phase, an amorphous phase, or a semi-crystalline phase.

5. The composition of claim 1, wherein the effective average particle size of the thiazepine particles is selected from the group consisting of less than 1900 nm, less than 1800 nm, less than 1700 nm, less than 1600 nm, less than 1500 nm, less than 1400 mm, less than 1300 nm, less than 1200 nm, less than 1100 nm, less than 1000 nm, less than 900 nm, less than 800 nm, less than 700 nm, less than 600 nm, less than 500 nm, less than 400 nm, less than 300 nm, less than 250 nm, less than 200 nm, less than 100 nm, less than 75 nm, and less than 50 nm.

6. The composition of claim 1, wherein the composition is formulated:
(a) for administration selected from the group consisting of parental injection, oral administration in solid, liquid, or aerosol form, vaginal, nasal, rectal, optically, ocular, local, buccal, intracisternal, intraperitoneal, and topical administration;
(b) into a dosage form selected from the group consisting of liquid dispersions, gels, sachets, solutions, aerosols, ointments, tablets, capsules, creams, and mixtures thereof.
(c) into a dosage form selected from the group consisting of controlled release formulations, fast melt formulations, lyophilized formulations, delayed release formulations, extended release formulations, pulsatile release formulations, and mixed immediate release and controlled release formulations; or
(d) any combination thereof.

7. The composition of claim 1, wherein the composition further comprises one or more pharmaceutically acceptable excipients, carriers, or a combination thereof.

8. The composition of claim 1, wherein:
(a) thiazepine is present in an amount consisting of from about 99.5% to about 0.001%, from about 95% to about 0.1%, and from about 90% to about 0.5%, by weight, based on the total combined weight of the thiazepine and at least one surface stabilizer, not including other excipients;
(b) at least one surface stabilizer is present in an amount of from about 0.5% to about 99.999% by weight, from about 5.0% to about 99.9% by weight, and from about 10% to about 99.5% by weight, based on the total combined dry weight of the thiazepine and at least one surface stabilizer, not including other excipients; or
(c) a combination of (a) and (b).

9. The composition of claim 1, wherein the surface stabilizer is selected from the group consisting of an ionic surface stabilizer, an anionic surface stabilizer, a cationic surface stabilizer, a zwitterionic surface stabilizer, and a non-ionic surface stabilizer.

10. The composition of claim 1, wherein the surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl P-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, lysozyme, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipid, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅-dimethyl hydroxyethyl ammonium chloride, C₁₂₋₁₅-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, polyquaternium 10, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, quaternized ammonium salt polymers, alkyl pyridinium salts; amines, amine salts, amine oxides, imide azolinium salts, protonated quaternary acrylamides, methylated quaternary polymers, and cationic guar.

11. The composition of claim 1, additionally comprising one or more active agents useful for the treatment of hypertension and related diseases.

12. The composition of claim 11, wherein the related disease is selected from the group consisting of ischemic heart disease, stroke, peripheral artery disease, hypertensive heart disease, and renal failure.

13. The composition of claim 11, wherein the one or more active agent is selected from the group consisting of diuretics, beta-blockers, ACE inhibitors, calcium channel blockers, alpha blockers, alpha-beta blockers, angiotensin antagonists, nervous system inhibitors, and vasodilators.

14. The composition of claim 1, wherein:
(a) upon administration to a mammal the thiazepine particles redisperse such that the particles have an effective average particle size selected from the group consisting of less than 2 microns, less than 1900 nm, less than 1800 nm, less than 1700 nm, less than 1600 nm, less than 1500 nm, less than 1400 nm, less than 1300 nm, less than 1200 nm, less than 1100 nm, less than 1000 nm, less than 900 nm, less than 800 nm, less than 700 nm, less than 600 nm, less than 500 nm, less than 400 nm, less than 300 nm, less than 250 nm, less than 200 nm, less than 150 nm, less than 100 nm, less than 75 nm, and less than 50 nm;
(b) the composition redisperses in a biorelevant media such that the thiazepine particles have an effective average particle size selected from the group consisting of less than 2 microns, less than 1900 nm, less than 1800 nm, less than 1700 nm, less than 1600 nm, less than 1500 nm, less than 1400 nm, less than 1300 nm, less than 1200 nm, less than 1100 nm, less than 1000 nm, less than 900 nm, less than 800 nm, less than 700 nm, less than 600 nm, less than 500 nm, less than 400 nm, less than 300 nm, less than 250 nm, less than 200 nm, less than 150 nm, less than 100 nm, less than 75 nm, and less than 50 nm; or
(c) a combination of (a) and (b).

15. The composition of claim 14, wherein the biorelevant media is selected from the group consisting of water, aqueous electrolyte solutions, aqueous solutions of a salt, aqueous solutions of an acid, aqueous solutions of a base, and combinations thereof.

16. The composition of claim 1, wherein:
(a) the Tₘₐₓ of the thiazepine, when assayed in the plasma of a mammalian subject following administration, is less than the Tₘₐₓ for a non-nanoparticulate composition of the same thiazepine, administered at the same dosage;
(b) the Cₘₐₓ of the thiazepine, when assayed in the plasma of a mammalian subject following administration, is greater than the Cₘₐₓ for a non-nanoparticulate composition of the same thiazepine, administered at the same dosage;
(c) the AUC of the thiazepine, when assayed in the plasma of a mammalian subject following administration, is greater than the AUC for a non-nanoparticulate composition of the same thiazepine, administered at the same dosage; or
(d) any combination thereof.

17. The composition of claim 16, wherein:
(a) the Tₘₐₓ is selected from the group consisting of not greater than 90%, not greater than 80%, not greater than 70%, not greater than 60%, not greater than 50%, not greater than 30%, not greater than 25%, not greater than 20%, not greater than 15%, not greater than 10%, and not greater than 5% of the Tₘₐₓ exhibited by a non-nanoparticulate composition of the same thiazepine, administered at the same dosage;
(b) the Cₘₐₓ is selected from the group consisting of at least 50%, at least 100%, at least 200%, at least 300%, at least 400%, at least 500%, at least 600%, at least 700%, at least 800%, at least 900%, at least 1000%, at least 1100%, at least 1200%, at least 1300%, at least 1400%, at least 1500%, at least 1600%, at least 1700%, at least 1800%, or at least 1900% greater than the Cₘₐₓ exhibited by a non-nanoparticulate composition of the same thiazepine, administered at the same dosage;
(c) the AUC is selected from the group consisting of at least 25%, at least 50%, at least 75%, at least 100%, at least 125%, at least 150%, at least 175%, at least 200%, at least 225%, at least 250%, at least 275%, at least 300%, at least 350%, at least 400%, at least 450%, at least 500%, at least 550%, at least 600%, at least 750%, at least 700%, at least 750%, at least 800%, at least 850%, at least 900%, at least 950%, at least 1000%, at least 1050%, at least 1100%, at least 1150%, or at least 1200% greater than the AUC exhibited by the non-nanoparticulate formulation of the same thiazepine, administered at the same dosage; or
(d) any combination thereof.

18. The composition of claim 1 which does not produce significantly different absorption levels when administered under fed as compared to fasting conditions.

19. The composition of claim 18, wherein the difference in absorption of the thiazepine, when administered in the fed versus the fasted state, is selected from the group consisting of less than 100%, less than 90%, less than 80%, less than 70%, less than 60%, less than 50%, less than 40%, less than 30%, less than 25%, less than 20%, less than 15%, less than 10%, less than 5%, and less than 3%.

20. The composition of claim 1, wherein administration of the composition to a human in a fasted state is bioequivalent to administration of the composition to a subject in a fed state.

21. The composition of claim 20, wherein “bioequivalency” is established by:
(a) a 90% Confidence Interval of between 0.80 and 1.25 for both Cₘₐₓ and AUC; or
(b) a 90% Confidence Interval of between 0.80 and 1.25 for AUC and a 90% Confidence Interval of between 0.70 to 1.43 for Cₘₐₓ.

22. A method of preparing a nanoparticulate anti-hypertensive active agent comprising contacting particles of a thiazepine compound having anti-hypertensive pharmaceutical properties with at least one surface stabilizer for a time and under conditions sufficient to provide a nanoparticulate thiazepine composition having an effective average particle size of less than 2000 nm.

23. The method of claim 22, wherein the thiazepine is temocapril or a salt thereof.

24. The method of claim 23, wherein the salt is the hydrochloride salt.

25. The method of claim 22, wherein the contacting comprises grinding, wet grinding, homogenization, freezing, template emulsion, precipitation, or a combination thereof.

26. The method of claim 22, wherein the effective average particle size of the thiazepine particles is selected from the group consisting of less than 1900 nm, less than 1800 nm, less than 1700 nm, less than 1600 nm, less than 1500 nm, less than 1000 nm, less than 1400 nm, less than 1300 nm, less than 1200 nm, less than 1100 nm, less than 900 nm, less than 800 nm, less than 700 nm, less than 600 nm, less than 500 nm, less than 400 nm, less than 300 nm, less than 250 nm, less than 200 nm, less than 100 nm, less than 75 nm, and less than 50 nm.

27. A method for treating hypertension or a related condition or disease comprising administering to a patient in need a composition comprising:
(a) particles of a thiazepine compound having anti-hypertensive pharmaceutical properties and having an effective average particle size of less than 2000 nm; and
(b) at least one surface stabilizer.

28. The method of claim 27, wherein the thiazepine is temocapril or a salt or derivative thereof.

29. The method of claim 28 wherein the salt is the hydrochloride salt.

30. The method of claim 27, wherein the related disease or condition is selected from the group consisting of ischemic heart disease, stroke, peripheral artery disease, hypertensive heart disease, and renal failure.

31. The method of claim 27, wherein the effective average particle size of the thiazepine particles is selected from the group consisting of less than 1900 nm, less than 1800 nm, less than 1700 nm, less than 1600 nm, less than 1500 nm, less than 1000 nm, less than 1400 nm, less than 1300 nm, less than 1200 nm, less than 1100 nm, less than 900 nm, less than 800 nm, less than 700 nm, less than 600 nm, less than 500 nm, less than 400 nm, less than 300 nm, less than 250 nm, less than 200 nm, less than 100 nm, less than 75 nm, and less than 50 nm.

32. The method of claim 27, wherein the thiazepine particles have improved bioavailability as compare to conventional non-nanoparticulate thiazepine particles.

33. A stable nanoparticulate anti-hypertensive pharmaceutical composition comprising:
(a) particles of a thiazepine compound having anti-hypertensive pharmaceutical properties and having an effective average particle size of about 2000 nm; and
(b) at least one surface stabilizer.

34. A method of preparing a nanoparticulate anti-hypertensive active agent comprising contacting particles of a thiazepine compound having anti-hypertensive pharmaceutical properties with at least one surface stabilizer for a time and under conditions sufficient to provide a nanoparticulate thiazepine composition having an effective average particle size of about 2000 nm.

35. A method for treating hypertension or a related condition or disease comprising administering to a patient in need a composition comprising:
(a) particles of a thiazepine compound having anti-hypertensive pharmaceutical properties and having an effective average particle size of about 2000 nm;
(b) at least one surface stabilizer.
